(19) United States
(12) Patent Application Publication
(10) Pub. No.: US 2024/0299690 A1
(43) Pub. Date: Sep. 12, 2024

(54) OBTURATORS AND TUBE ASSEMBLIES

(71) Applicant: SMITHS MEDICAL INTERNATIONAL LIMITED

(72) Inventors: Timothy Bateman, Hythe (GB); Andrew Thomas Jeffrey, Romney Marsh (GB); Christopher John Woosnam, London (GB)

(73) Assignee: SMITHS MEDICAL INTERNATIONAL LIMITED, Ashford (GB)

(21) Appl. No.: 18/281,643

(22) PCT Filed: Mar. 11, 2022
(86) PCT No.: PCT/GB2022/000030

§ 371 Date: Sep. 12, 2023

(30) Foreign Application Priority Data
Mar. 25, 2021 (GB) 2104206.4

(51) Int. Cl. A61M 16/04

(52) U.S. Cl. CPC A61M 16/0472

(57) ABSTRACT

An obturator (2) for a tracheostomy tube (1) has a shaft (20) with a bullet-shape nose (24) at its patient end and a clip (21) at its machine end with arms (34) engaging recesses (15) on a coupling (13) at the machine end of the tube (1). The clip (21) is rotatably mounted with the shaft (20) by means of an engaging spigot (135) and aperture (213) and is retained angularly by dimples (219) on the clip engaged with recesses (141) on the obturator shaft. The obturator (2) is released from the tube (1) by twisting the clip against the retention of the dimples with the recesses and without applying any twisting force to the patient end of the obturator or tube.

This invention relates to obturators of the kind for a medical tube, the obturator having a patient end shaped with a tapered nose arranged to protrude from the patient end of the tube, a machine end adapted to fasten with the machine end of the tube and a shaft extending between the patient and machine end of the obturator, the machine end of the obturator being in the form of a clip releasable from engagement with the machine end of the tube by twisting relative to the tube.

The invention is more particularly concerned with obturators for use in tracheostomy tubes.

Tracheostomy tubes are often inserted with the aid of an obturator having a pointed end projecting from the patient end of the tube. The tip of the obturator helps separate tissue, enabling smooth entry of the tube. The obturator also helps stiffen the tube and prevents ingress of tissue into the tube, which could cause blockage. Examples of tracheostomy obturators are described in U.S. Pat. Nos. 4,246,897, 5,222,487, GB2224213, GB2316321 and GB2341102. Any displacement of the obturator from its correct position may make insertion of the tube more difficult and, by reducing the smoothness of the patient end of the assembly, may cause trauma to tissue around the stoma. After insertion, the obturator is pulled out of the machine end of the tube. GB 2316321 describes an obturator with an integral machine end that clips onto the coupling of the tracheostomy tube, the clip being released by twisting it relative to the tube. In this arrangement, the main body of the obturator is a strap of rectangular section, so it is relatively flexible to enable twisting. U.S. Pat. No. 6,481,436 describes an obturator with a similar clip but where the machine end portion of the shaft of the obturator is shaped to make it more flexible than the patient end of the obturator. This enables the clip to be twisted to release it from the tube more easily. However, with both forms of obturator the action of twisting the clip does result in some torque being applied to the patient end of the obturator and this can result in some twisting movement of the tube itself. This twisting movement of the tube within the trachea can cause discomfort and may cause tissue damage.

It is an object of the present invention to provide an alternative obturator and tube assembly.

According to one aspect of the present invention there is provided an obturator of the above-specified kind, characterised in that the clip and shaft are separately formed components, and that the clip is rotatably mounted with the machine end of the shaft for at least limited rotation relative to the shaft about the longitudinal axis of the shaft so that the clip can be rotated to release it from engagement with the machine end of the tube without applying any substantial torque to the shaft.

The clip preferably includes a central aperture, the machine end of the shaft including a spigot rotatably received in the aperture in the clip. The machine end of the shaft and the clip may have cooperating detents that retain the clip angularly relative to the shaft until sufficient force is applied to rotate the clip relative to the shaft. The detents may include a projection on the clip and a recess on the machine end of the shaft. The machine end of the obturator preferably includes two resilient arms each having a catch at its patient end shaped to engage a lip on the outside of the machine end of the tube.

According to another aspect of the present invention there is provided an assembly including a medical tube and an obturator according to the above one aspect of the present invention fitted in the tube.

The tube is preferably a tracheostomy tube.

A tracheostomy tube assembly including an obturator according to the present invention, will now be described, by way of example, with reference to the accompanying drawings, in which:

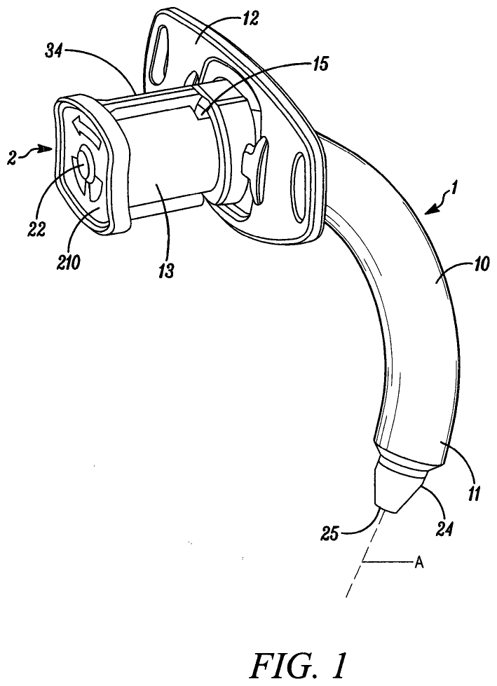

FIG. 1 is a perspective view of the assembly with the obturator in its engaged state;

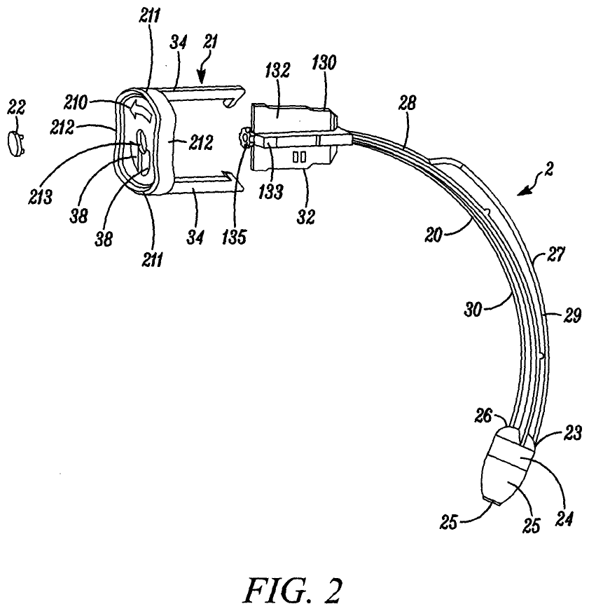

FIG. 2 is a perspective view of the obturator with its components separated;

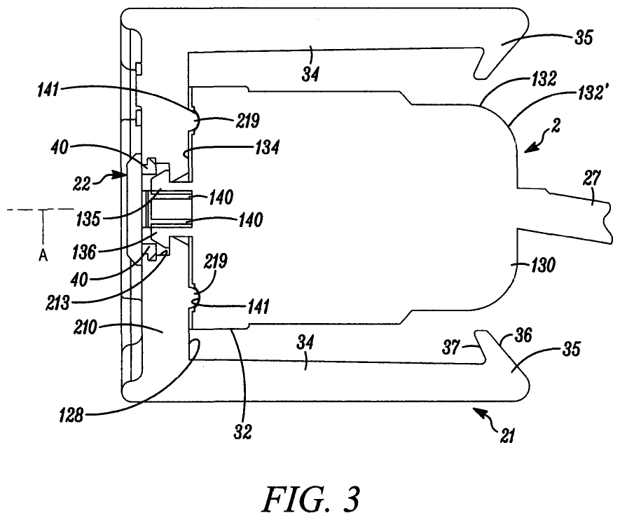

FIG. 3 is an enlarged cross-sectional side elevation view of the machine end of the obturator;

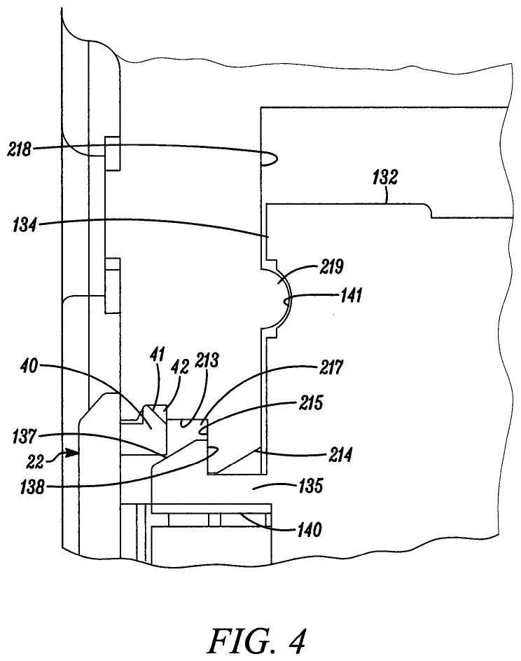

FIG. 4 is a further enlarged cross-sectional view of a part of the machine end of the obturator; and

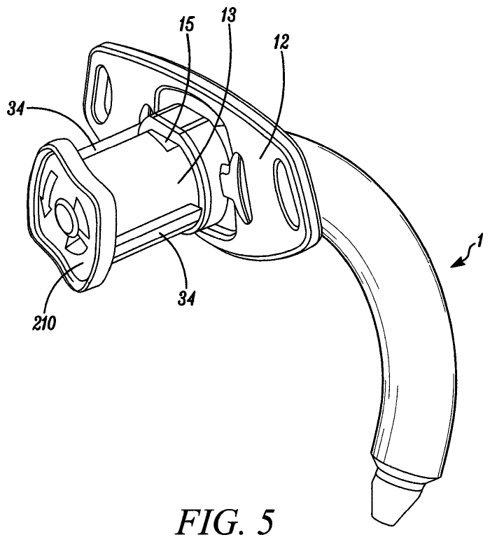

FIG. 5 is perspective view of the assembly with the obturator in its released state.

With reference first to FIGS. 1 and 2, the assembly comprises a tracheostomy tube 1 and an obturator 2.

The tube 1 has a conventional shaft 10 of circular section, which is moulded with a curved shape and a constant radius of curvature along its length. The patient end 11 of the shaft 10 is cut square and tapered around its outer surface to be atraumatic. At its machine end, the shaft 10 is moulded integrally with a flange 12 by which the tube can be secured to the patient's neck. The machine end of the shaft 10 is also bonded to a coupling 13 having a male tapered outer surface shaped to receive a female tapered coupling (not shown) connected to a patient ventilation or anaesthetic circuit. Alternatively, where the patient is breathing spontaneously, the coupling 13 is left open. Externally, the coupling 13 is of substantially cylindrical shape apart from surface formations provided by two, short undercut lips 15 located diametrically opposite one another at the patient end of the coupling. The shaft 10 is shown without any sealing cuff but it will be understood that the tube could have a conventional sealing cuff and other conventional features such as a suction lumen opening above the cuff by which secretions can be collected.

The obturator 2 comprises three separate components, namely a shaft 20, a retaining clip 21 and an optional cap 22. The shaft 20 is a single-piece, integral moulding of a stiff plastics material such as high-density polyethylene. At its patient end 23, the shaft 20 has a bullet-shape nose 24, which is a close fit within the patient end 11 of the tube 1. The nose 24 has a tapered tip 25, which, in use, projects from the tube 1 so as to form a pointed continuation of the patient end of the shaft 10. A small diameter bore (not visible) extends along the nose 24 from its tip 25 to an opening 26 at its rear end.

Extending rearwardly from the nose 24 the shaft 20 has a curved strap 27 moulded to the curvature of the tube 1. Typically, the strap 27 is about 80 mm long. The strap 27 comprises a web 28 of substantially rectangular section arranged to extend diametrically of the tube shaft 10 as a close sliding fit along its entire length. The strap 27 may also have an additional web 29 extending along the major part of the length of the strap. This additional web 29 extends radially and orthogonally of the first web 28 projecting from one side only towards the outside of its curve so that it substantially contacts the inner wall of the shaft 10 of the tube 1 on the outside of its curvature. The major part of the strap 27 from its patient end, therefore, has a substantially T-shape in section, with just a shallow rib 30 along the side opposite the additional web 29 and is substantially rigid.

The machine end 30 of the strap 27 has a substantially rectangular shape in section. The strap 27 could have various different shapes and could have a cruciform shape along its entire length, or a T shape, or it could be a flat rectangular plate along its length. The strap could have projections (not shown) spaced along its surface, such as of semi-circular shape, to centre the obturator 2 within the bore of the tube 1.

With reference now also to FIGS. 3 and 4, the machine end 32 of the strap 27 has an enlarged, cruciform section 130 formed integrally as a single part with the strap. The cruciform section 130 comprises a vertical plate 132 and a horizontal plate 133. The machine end 32 has a tapered exterior, which is a close friction fit within the coupling 13 of the tube 1. Centrally of the machine end face 134 of the enlarged section 130 projects an integral, short, hollow spigot 135 of circular section. The spigot 135 is formed with a barb 136 at its free end comprising an enlarged tapered flange 137 forming a radial step 138 around its forward, patient-facing end. The step 138 is spaced a short distance rearwardly from the rear edge 134 of the plates 132 and 133. The spigot 135 is segmented by several longitudinal axial slots 140 to make it compliant radially. The rear edge of one of the plates 132 has two shallow concave recesses 141 equally spaced on opposite sides of the spigot 135 towards the outer edge of the plate.

The clip 21 is fitted securely on the rear end of the shaft 20 in such a way that it can be rotated or angularly displaced about the axis of the spigot 135 coincident with the longitudinal axis “A” of the shaft. The clip 21 comprises a flat, radially extending flange or grip 210 with a generally square shape having two opposite convex curved sides 211 and two opposite concave sides 212, which provide purchase for gripping between the finger and thumb to enable the clip to be gripped, rotated, and pulled. The flange or grip 210 has a central aperture 213 extending through its thickness. The aperture 213 is profiled at its forward end with a tapered portion 214 reducing in diameter rearwardly to a minimum diameter equal to the external diameter of the spigot 135 on the rear end of the strap 27 forwardly of its barb 136. To the rear of the tapered portion 214 the aperture 213 is shaped with an annular step 215, which faces to the rear and is bounded by a short outer cylindrical wall 216 forming a recess 217 in which the barb 136 can be received. The dimensions and profile of the forward end of the aperture 213 are such that when the clip 21 is assembled by pushing on to the shaft 20 the taper 137 around the outside of the spigot 135 slides along the taper 214 of the aperture. This causes the free end of the spigot 135 to be resiliently compressed inwardly until the barb 136 snaps into the recess 217 and its forward-facing step 138 locates against the rear-facing step 215 in the aperture 213. This prevents the clip 21 being removed from the shaft 20 after assembly.

The forward-facing surface 218 of the flange 210 on the clip 21 is moulded with two shallow, projections in the form of dimples 219 positioned diametrically opposite one another at a radial distance where they will align and locate with the recesses 141 on the rear edge 134 of the plate 132 when the clip is appropriately aligned with the shaft 20. The dimples 219 and recesses 141 thereby form detents that provide a tactile click to indicate when the clip 21 is correctly angular aligned with the shaft 20. They also help retain the clip 21 in correct alignment until sufficient rotational force is applied it to overcome the retaining force provided by the detents. The spigot 135 and aperture 213 provide a rotatable coupling between the clip 21 and the shaft 20 of the obturator 2 so that the clip can be twisted about the longitudinal axis “A” of the obturator without applying any significant torque to its shaft and hence to the patient end 11 of the tube 1.

The clip 21 also includes two integral resilient arms 34 that are moulded with and extend forwardly from the flange 210 on opposite sides of the obturator 2. The arms 34 are terminated at their patient end by surface formations in the form of inwardly directed catches 35 shaped with an inclined ramp 36 and a ledge 37. The dimensions of the arms 34 are such that, when the flange 210 abuts the machine end of the coupling 13, the arms extend along opposite sides of the coupling with the catches 35 engaging under the lips 15, thereby preventing the obturator 2 being removed from the tube. Two vent holes 38 extend through the flange 210 in alignment with corners between the two plates 132 and 133.

The obturator 2 is completed by an optional small circular cap or cover 22. The forward surface of the cap 22 has several small catches 40 projecting forwardly and spaced around it close to its edge. Each catch 40 has an outwardly-projecting tooth 41 shaped to engage in an annular groove 42 formed in the aperture 213 of the clip 21 close to its rear end and thereby retain the cap 22 on the clip.

In use, the obturator 2 is pushed fully into the tube 1 so that its nose 24 projects from the patient end 11 of the tube and so that the catches 34 engage the lips 15 on the coupling 13, thereby fastening the machine end of the obturator with the machine end of the tube. The assembly is inserted into the patient in the usual way, the obturator 2 providing a tapered lead for the tube 10 into the tracheostomy. The cross-sectional shape of the obturator strap 27 is selected to give it the desired stiffness for the type of patient. Rearward movement of the obturator 2 relative to the tube 1 is prevented by engagement of the catches 34 with the lips 15. The patient can breathe through the assembly during insertion because of the bore through the nose 24, the passage between the shaft 20 of the obturator and the inside of the tube 1, and the holes 38 in the flange 210. If desired, a guidewire (not shown) could be threaded through the bore in the nose 24 prior to insertion and the assembly slid into position along the guidewire. In order to reduce the risk that the rear end of the guidewire catches on the vertical plate 132 when the guidewire is inserted from the patient end of the obturator, the leading edge 132′ of the vertical plate is rounded at its edge. When the assembly has been inserted to the correct location, the obturator 2 is removed by gripping the flange 210 and twisting it through about 20°, to the position shown in FIG. 5, so that the catches 34 disengage the lips 15. This enables the catches 34 and hence the obturator 2 to be pulled rearwardly of the coupling 13.

The rotatable connection between the clip 21 and the shaft 20 of the obturator 2 enables the flange 210 to be twisted easily, thereby releasing the obturator without twisting the patient end of the obturator and without applying any significant torque to the shaft 10 of the tracheostomy tube 1. This avoids, or substantially reduces, any uncomfortable or damaging movement of the tube 1 in the patient. After the obturator 2 has been removed, the coupling 13 can be connected to a ventilation circuit or left open, in the usual way.

It will be appreciated that alternative rotatable connections could be used between the clip and the shaft of the obturator. The invention could be used with tubes, other than tracheal tubes, where it is necessary to prevent displacement of an obturator relative to a tube.

1-7. (canceled)

8. An obturator for a medical tube having a patient end and a machine end, the obturator having a patient end shaped with a tapered nose arranged to protrude from the patient end of the tube, a machine end adapted to fasten with the machine end of the tube and a shaft extending between the patient end and the machine end of the obturator, the machine end of the obturator being in the form of a clip releasable from engagement with the machine end of the tube by twisting relative to the tube, characterised in that the clip and the shaft are separately formed components, and that the clip is rotatably mounted with the machine end of the shaft for at least limited rotation relative to the shaft about the longitudinal axis of the shaft so that the clip can be rotated to release it from engagement with the machine end of the tube without applying any substantial torque to the shaft.

9. The obturator according to claim 8, characterised in that the clip includes a central aperture and that the machine end of the shaft includes a spigot rotatably received in the aperture in the clip.

10. The obturator according to claim 8, characterised in that the machine end of the shaft and the clip have cooperating detents that retain the clip angularly relative to the shaft until sufficient force is applied to rotate the clip relative to the shaft.

11. The obturator according to claim 10, characterised in that the detents include a projection on the clip and a recess on the machine end of the shaft.

12. The obturator according claim 8, characterised in that the machine end of the obturator includes two resilient arms each having a catch at its patient end shaped to engage a lip on the outside of the machine end of the tube.

13. An assembly including a medical tube and an obturator fitted in the tube, the tube having a patient end and a machine end, the obturator having a patient end shaped with a tapered nose arranged to protrude from the patient end of the tube, a machine end adapted to fasten with the machine end of the tube and a shaft extending between the patient end and the machine end of the obturator, the machine end of the obturator being in the form of a clip releasable from engagement with the machine end of the tube by twisting relative to the tube, characterised in that the clip and shaft are separately formed components, and that the clip is rotatably mounted with the machine end of the shaft for at least limited rotation relative to the shaft about the longitudinal axis of the shaft so that the clip can be rotated to release it from engagement with the machine end of the tube without applying any substantial torque to the shaft.

14. The assembly according to claim 13, wherein the tube is a tracheostomy tube.
